(19) United States
(12) Patent Application Publication
(10) Pub. No.: US 2026/0102210 A1
(43) Pub. Date: Apr. 16, 2026

(54) SURGICAL RECONSTRUCTION PLANNING AND PERFORMANCE PROCESS

(71) Applicant: ReconRoadmap LLC, Dayton, OH (US)

(72) Inventors: Heidi Baca Donnelly, Dayton, OH (US); Kimberly Rachel Shao, Denver, CO (US)

(73) Assignee: ReconRoadmap LLC, Dayton, OH (US)

(21) Appl. No.: 19/354,054

(22) Filed: Oct. 9, 2025

Related U.S. Application Data
(60) Provisional application No. 63/705,758, filed on Oct. 10, 2024.

(51) Int. Cl. A61B 34/10; A61B 90/00; G16H 20/40

(52) U.S. Cl. CPC A61B 34/10; A61B 90/361; G16H 20/40; A61B 2090/364; A61B 2090/373

(57) ABSTRACT

Current embodiments include online platforms and methods for practicing an at least partially automated planning tool for surgically reconstructing a cutaneous defect; a surgeon or the platform itself selects from multiple reconstructive options depending on one or more relevant factors, and electronically stored information related to such reconstructive option is presented in the form of diagrams, before and after photographs, videos, and procedural execution tips; various embodiments enable creation of patient-specific projects in which one or more visual objects appear in a preview window viewable by the surgeon, enabling the project to be saved, opened and reopened, and modified by changing the visual objects in the project; in use, smart glasses can be paired to the system for access and real-time viewing of the surgical plan from a patient-specific project through a display integrated with the smart glasses.

### CROSS-REFERENCE TO RELATED APPLICATIONS

This patent application claims the benefit of and priority to U.S. Provisional Patent Application Ser. No. 63/705,758 with a filing date of Oct. 10, 2024, the contents of which are fully incorporated herein by reference.

### STATEMENT OF GOVERNMENT INTEREST

None.

### FIELD OF INVENTION

A novel and advantageous surgical reconstruction planning system provides efficient access to a comprehensive and expanding compilation of resources, allowing users to search by parameters not currently available permitting intraoperative use during reconstructive surgery, and iteratively modified in real-time.

### BACKGROUND

Repair of a cutaneous defect after a skin cancer removal is one example of reconstructive surgery. In this type of surgery, typically a surgeon relies on what he or she learned in medical training or one's recollection of reference articles and similar resources, or a surgeon laboriously obtains relevant information online or from hardcopy journals, textbooks, and video tutorials. There is no easy and efficient way for the surgeon to access a comprehensive list of all possible reconstructive options for a specific defect. Additionally, surgeons are currently unable to narrow their search by certain parameters such as defect size and depth. Consequently, valuable time is spent searching for such resources and options. Currently, a surgeon wanting to search options for repairing a patient defect must access multiple textbooks, articles from various journals, online video tutorials, or some combination of these. Finding an appropriate journal article is time-consuming and challenging as the names of the articles don't allow for a user to search based on size of the defect, depth, or sub-anatomic unit. Textbooks are usually very broad outlines and again don't usually include innovative flap designs. To find a relevant video, a surgeon would have to know the exact name of the flap he/she wants to use. Additionally, if the surgeon is planning based on memory alone, multiple possible repair options, especially novel ones, may be missed. These alternatives could have improved the patient's result.

Further, such resources generally are spread across multiple platforms. These may include but would not be limited to books, online video tutorials, journals, professional conference notes, and the like. Currently, there is no single platform that allows users to access vetted information in the planning for these surgeries, much less intraoperatively, with such planning information being specific to a patient's defect.

This is notable because surgical plans must be tailored to the individual. A surgeon must be able to create the surgical plan, consider it with the patient through consultation, and make adjustments based on patient decisions or continued gathering of information.

A surgeon must consider the range of factors that will determine which reconstructive options may be available to use on any given case. A defect in need of repair can be defined by location, size, depth, and other factors. Different body parts require different surgical approaches. Indeed, consider one body part, the lip, and the number of anatomic subunits where defects can be present (philtrum, upper and lower cutaneous lip, upper and lower vermilion, oral commissure, nasal sill, etc.), as well as sub-subunits in some cases, such as the inferior philtrum and superior philtrum.

Instead of surgeons searching through textbooks, online journals and video tutorials when developing a surgical plan, there is a need in the medical field for surgeons to have access to an online collaborative encyclopedia of cutaneous reconstruction, allowing him or her to search according to the above factors, and providing ready access to a curated list of reconstructive options with related diagrams, photos (i.e., photographs), videos, and execution tips.

In addition, there is a need in the medical field for such a resource to be changeable, both as the knowledge base and use of surgical techniques grow, and for individual surgeons to be able to create, save, and modify surgical plans for particular patients. Such plans, which in the description of embodiments herein also are referred to as “projects,” should be available to the surgeon prior to reconstructive surgery to discuss options with his or her patient, and intraoperatively as a real time resource for the surgeon. Generally, surgeons lack the time, both pre-operatively and intraoperatively, to research new or alternative ways to repair a cutaneous defect. Currently, to the inventors'knowledge, there are no online platforms or resources that address cutaneous reconstruction in this way, providing timely access to at least a partially automated method for planning a surgical reconstruction of a cutaneous defect in which a surgeon may select from multiple reconstructive options depending on one or more relevant factors. Therefore, the present disclosure provides a more efficient process for the surgeon to plan, adjust the plan, and access and perform a complete array of closure options and curated execution tips.

These and other advantages and features of the embodiments provided for herein are aimed at improving patient care and surgical outcomes, as will be evident from the following descriptions.

### SUMMARY OF EMBODIMENTS

Present embodiments include a system for surgeons, and those working for them and otherwise assisting in pre-planning for reconstructive surgeries, which helps identify particular flaps (cutaneous repairs) that could be used for a certain defect, based on findings specific to a patient. This curated list of possible flaps can then be used to guide consultation with a surgeon's patient to decide upon a specific repair. Once a particular flap is selected, the system access provides additional details about that flap so the surgeon can plan the surgery from a single resource. Such a system overcomes the problems associated with the existence of hundreds of potential flaps that can be employed across numerous anatomic units, as well as the current problem associated with having to look in many different places for comprehensive resources to plan a case, a process that conventionally has proven laborious and time-consuming. The system presented also will include ways for users to ask expert surgeons questions regarding the details and execution of each flap.

### BRIEF DESCRIPTION OF THE FIGURES

The drawings, schematics, figures, and descriptions herein are to be understood as illustrative of structures, features and aspects of the present embodiments and do not limit the scope of the embodiments. Where the figures provide or suggest dimensional information, the scope of the application is not limited to the precise arrangements, scales, or dimensions as shown in the drawings, nor as discussed in the textual descriptions.

FIG. 1 is an illustration of a head frontal view image with anatomic units that a surgeon can click on for more information, according to a range of embodiments provided herein.

FIG. 2A is an illustration of a head oblique view, according to the embodiments provided herein. FIG. 2B is a screen shot of a search bar with options (specifically anatomic subunits) to select, according to a range of embodiments provided herein.

FIG. 3A is an illustration of a head oblique view as in FIG. 2A, according to the embodiments provided herein. FIG. 3B is a screen shot of a landing page, following selection of the anatomic subunit, where measurements and parameters can be entered about a particular defect in need of surgical reconstruction, according to a range embodiments provided herein.

FIG. 4 provides a partial list of flap/cutaneous repair options available for a surgeon to search and select from, according to a range of embodiments provided herein.

FIG. 5 is a flowchart describing a process for performing an inclusion / exclusion selection process for selection of a repair option, based on the parameters the user chooses, according to a range of embodiments provided herein.

FIG. 6A is a diagram showing the face in which the solid black dots represent the anatomic sub-subunits which a specific flap could be used for, according to a range of embodiments provided herein.

FIG. 6B is an exemplary screen shot of subject matter available about a particular flap that a user might select, according to a range of embodiments provided herein.

FIG. 7A provides an example of a curated list of repair options, according to a range of embodiments provided herein. FIG. 7B provides a different example of a curated list of repair options, according to a range of embodiments provided herein.

FIG. 8 (a single figure divided into 8A-8G for better clarity and due to size of the figure as a whole) is a flow diagram showing how a user would incorporate the system of the present embodiments into a regular work flow, according to a range of embodiments provided herein.

FIG. 9A illustrates a landing page where a user may pose questions about a specific flap to an expert surgeon, according to a range of embodiments provided herein. FIG. 9B illustrates a landing page where an expert may respond to the questions posed.

### MULTIPLE EMBODIMENTS AND ALTERNATIVES

The present disclosure describes multiple embodiments and alternatives of a system which is directed to a comprehensive single platform of surgical resources to enhance and facilitate the roles of surgeons, patients, and medical assistants when determining the best surgical plan for a specific cutaneous reconstructive surgery. Such a platform may be in the form of an app available over a network, a website, or other system connected with stored information. When a surgeon (in this disclosure, “surgeon” may include those under the surgeon's direction, such as but not limited to residents, fellows, medical students and assistants) is able to gather and input specific details about a patient's surgical defect and obtain a populated list of appropriate repair options. It serves as a first resource enabling him or her to begin defining the surgical plan. For example, the populated list will include photographs or renderings of the repair options. Before and after photographs from prior cases may be made available with appropriate patient consent, which can serve as a patient resource to help guide the discussion of advantages and disadvantages for a particular surgical plan, particularly when a patient can visualize each repair option. In consultation, an informed patient surgeon can then choose the favored repair choice. Alternatively, with this resource even a surgical staff member could discuss repair options with a patient and inform a surgeon of the decision. This could ultimately save the surgeon even more time.

Following this choice, the embodiments of the present disclosure provide the surgeon access to detailed information about the selected repair that includes photos, anatomic and other diagrams, videos with step-by-step indications for how to perform a particular reconstructive option, expert tips, key suture, undermining plane, advantages, disadvantages, and links to reference articles. Such diagrams and videos can be displayed on an appropriate monitor or other device and used as an intraoperative resource for the surgeon. Optionally, a surgeon using smart glasses will be able to pair the smart glasses device to the system in order to access the surgical plan prepared for a patient, including these diagrams and videos with execution steps and tips as a virtual screen or transparent display viewable by the surgeon intraoperatively in real time to assist in preparing and executing the repair. It will be appreciated that smart glasses are only one display tool, and there could be others employed for a surgeon to access the system efficiently including during a surgery.

Notably, the embodiments of the present disclosure enable surgeons to organize repair options into projects, which will serve as their personal resource that can be accessed quickly and/or adjusted for a future surgical repair. For example, a surgeon can view diagrams to help them plan a flap design and access video resources to help in executing a difficult component of the flap, either in advance of surgery or in real-time during surgery.

For access to the system, surgeons may log in through a website or a smart phone application available over WiFi, cellular, satellite, or similar communication options. Such access may be through a paid subscription that uses typical login criteria such as, but not limited to, a username/password and e-mail address.

In some embodiments, an exemplary platform provides access to first electronically stored information, which is stored in memory operatively connected to the platform and comprises a list including one or more reconstructive options for a specific cutaneous defect. The platform then provides for user selection of a reconstructive option, which may be followed by a display of textual or graphic information about the selected reconstructive option, in addition to stored visual objects representing second electronically stored information, which may include, for example, photos and videos (e.g., anatomic photographs, surgical procedure videos), diagrams, instructions, tips, annotations, or other subject matter related to the selected surgical reconstructive option. The platform further provides for user selection of one or more visual objects from the set of second electronically stored information, and the platform is configured to arrange such visual object(s) in a preview window viewable by the user according to a first arrangement, wherein said arrangement includes a sequence in which the visual objects appear. In some embodiments, arranging the visual objects in a hierarchical data structure, for example by type of defect and format of visual objects to select from. This arrangement can be saved as a stored electronic file referred to as a project. An exemplary platform according to multiple embodiments and alternatives then enables the saved project to be opened and modified into subsequent arrangements, by at least one of the following: adding visual objects to the project, removing visual objects from the project, rearranging the sequence of visual objects in the project, and adding patient-specific measurements and other annotations regarding the procedure, marking certain ReconRoadmap™ photographic or video images as favorites, or adding details and notes related to the project.

As an alternative to a manual selection of a reconstructive option, in some embodiments, user input is accepted in the form of one or more factors related to a specific patient's cutaneous defect. Based on these factors, the platform executes machine readable program instructions to access the first electronically stored information. The system is configured to automatically select at least one reconstructive option for repairing the cutaneous defect from the first electronically stored information, and to save this selection as a project. Optionally, such a system and method can be further configured to associate the at least one identified reconstructive option with the one or more visual objects to be included in the project, to arrange the one or more visual objects in a sequence for display in the preview window, and to accompany this display textual or graphic information about the selected reconstructive option. In turn, a user can access and modify the saved project in the same manner as when the project is created and saved manually, as described above.

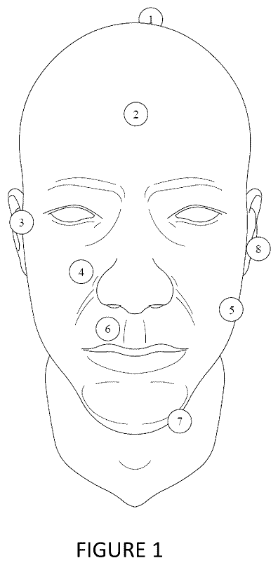

The following use case is not meant to be limiting, but can be used to illustrate the operation of the system and the way surgical plans can be created, used, and modified. A patient presents to the surgeon with a surgical defect, and the surgeon opens and logs into the system (aka ReconRoadmap™ with their username and password). The surgeon clicks on the anatomic area of the image on the head that corresponds to the location of the patient's defect, for example as illustrated in FIG. 1. FIG. 1 shows an image of a head that a surgeon can click on to choose the anatomic unit. In the figure, the number 1 corresponds with the scalp, the number 2 corresponds with the forehead, the number 3 corresponds with the peri-orbital area, the number 4 corresponds with the nose, the number 5 corresponds with the cheek, the number 6 corresponds with the lip, the number 7 corresponds with the chin, and the number 8 corresponds with the ear.

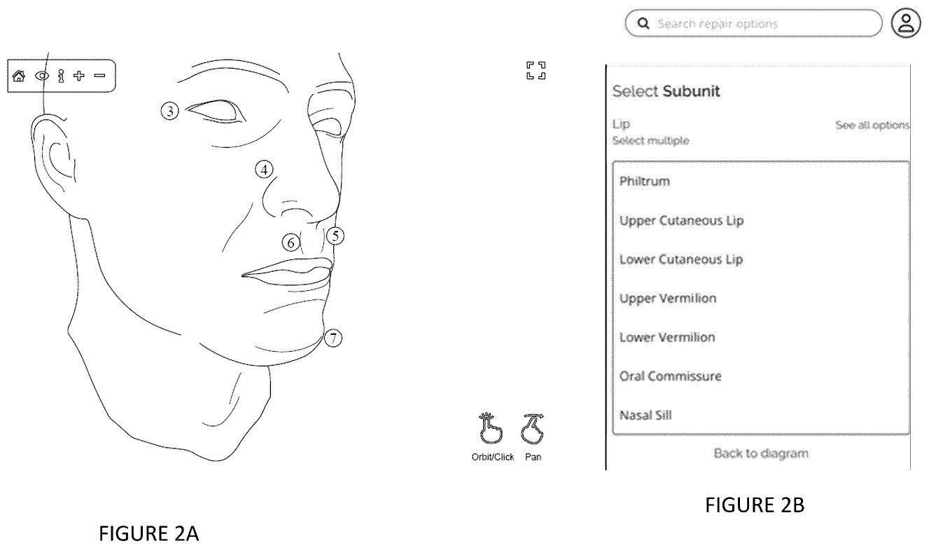

In turn, FIG. 2A shows the ability to rotate or pan that image for the selection of a specific anatomic unit, in this case the lip (corresponding with number 6 in FIG. 1). Upon selecting the anatomic unit, a pop-up appears as shown in FIG. 2B that lists subunits (i.e., sub-anatomic unit) of the selected anatomic unit. For example, when “lip” is selected as the anatomic unit (e.g., lip), the pop-up may list: philtrum, upper cutaneous lip, lower cutaneous lip, upper vermilion, lower vermilion, oral commissure, and nasal sill, as shown in FIG. 2B. Optionally, a search for a repair option for a particular defect by name can be performed, as seen in the search bar (i.e., “Search repair options”) that accompanies FIG. 2B.

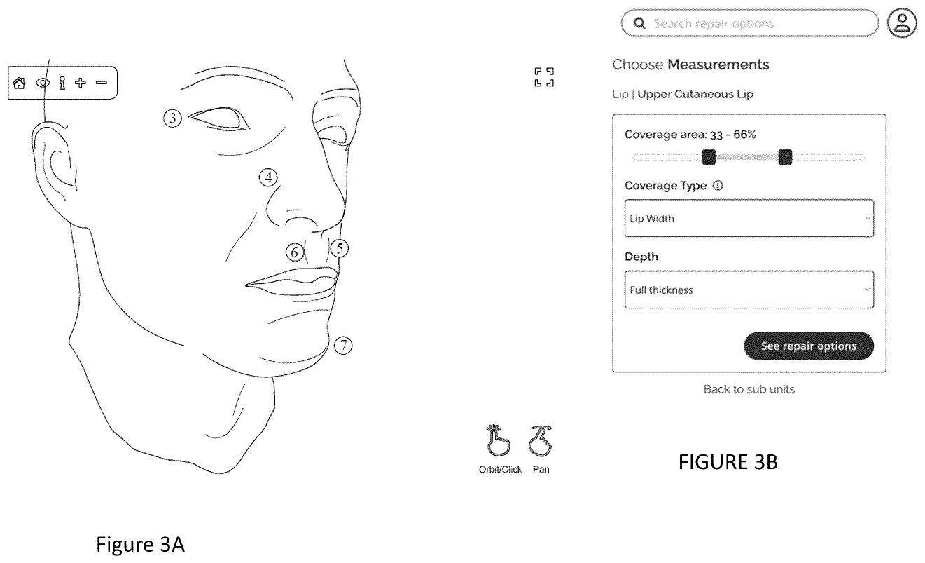

Once the selection of an appropriate sub-anatomic unit is made, the surgeon can then input the specific details about the patient's defect, with respect to the anatomic areas as shown in FIG. 3A, associated with the specific detail input as shown in FIG. 3B (shown to the right of the image which is FIG. 3A). For example, after a surgeon inputs or selects an anatomic subunit (e.g., “upper cutaneous lip” in this figure), a window appears (FIG. 3B) and a user can add specific parameters and measurements. In this example, a surgeon has chosen the lip as the anatomic unit, the upper cutaneous lip as subunit, and a full thickness defect measuring between 33-66% of the lip width. In some embodiments, as shown in FIG. 3B, a slider bar is provided as part of the user interface to allow a surgeon to specify the percent of coverage area.

These details may include, for example, the size of the defect (e.g. how much of the sub-anatomic unit is affected), coverage type, and the depth of the defect (e.g., how deep the defect extends). In some embodiments, also as shown in FIG. 3B, drop down boxes with predetermined options are provided to select coverage type and depth.

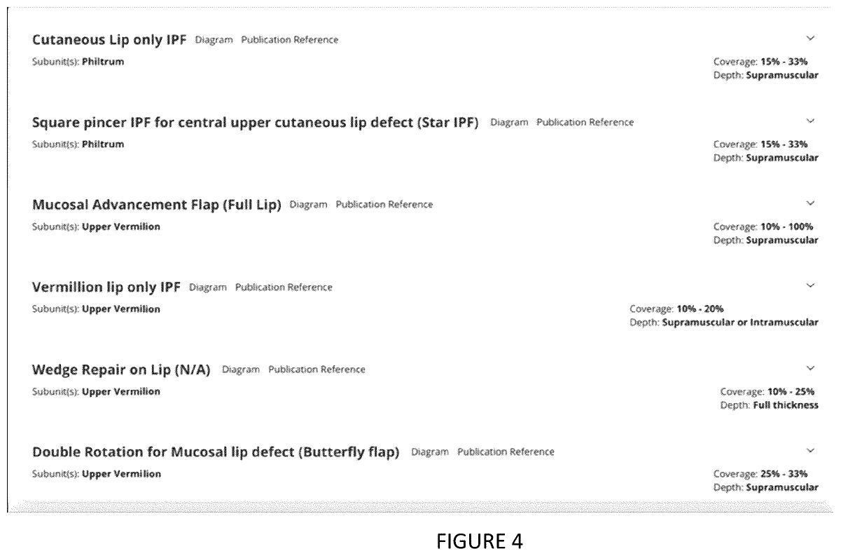

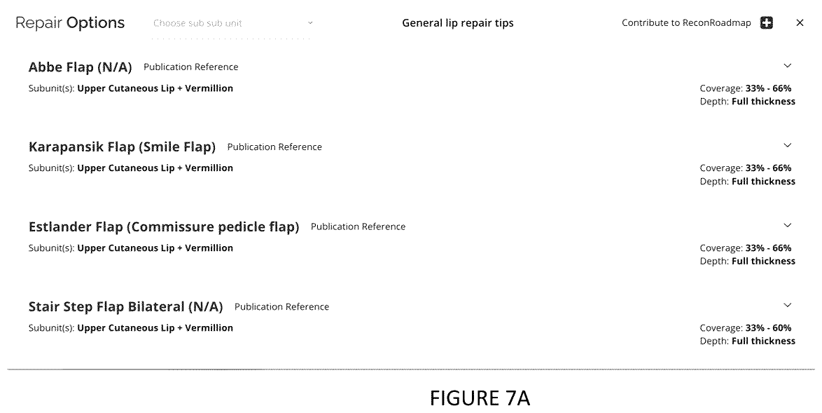

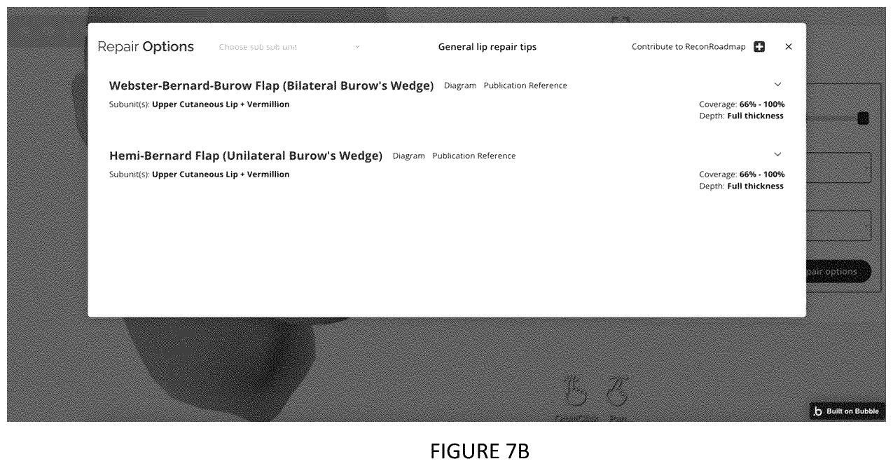

As an alternative to using the selection features described in FIGS. 2A, 2B, 3A, and 3B, the surgeon can be presented a comprehensive list of repair options (sometimes referred to herein as flaps), an excerpt of which is shown in FIG. 4. The surgeon is shown a table listing possible repair options. By searching or scrolling, for example, he or she can manually decide which flap he or she may want more information about. Clicking on a selection provides repair options and published references from relevant literature about the repair of particular anatomic subunits. Similar excerpts matching a particular repair option to findings are shown in FIGS. 7A, 7B.

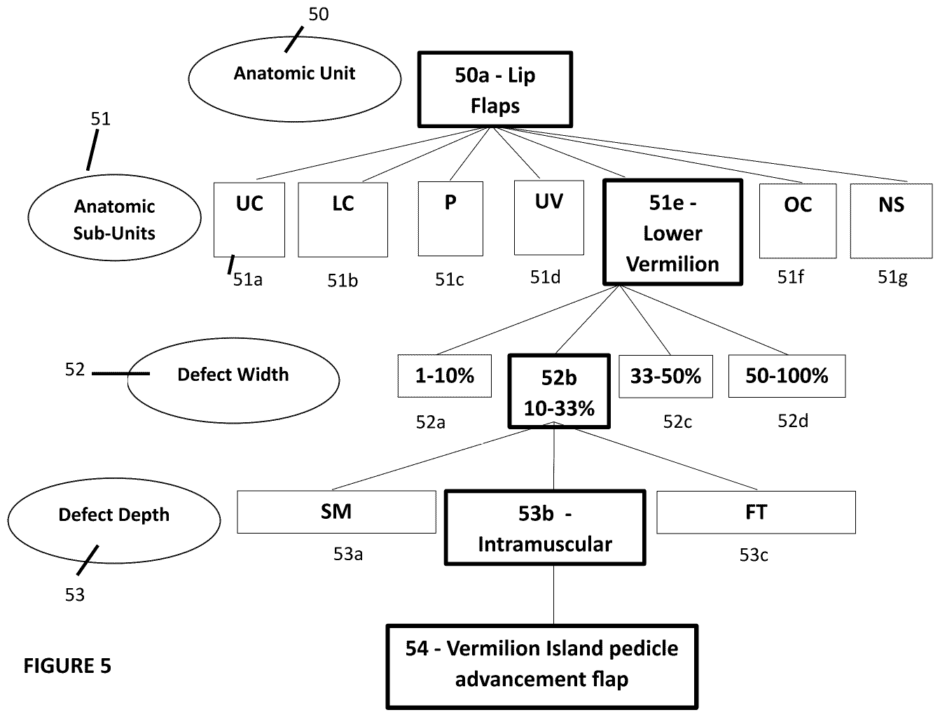

To further illustrate the inventive principles herein by which a repair option can be selected by the surgeon, FIG. 5 is a flowchart for identifying a suitable flap to perform, which indicates how the inventive system is able to provide an exemplary and hierarchical process to include or exclude repair options, based on the patient's details. In the figure, ovals arranged as rows labeled 50 (anatomic unit), 51 (anatomic subunits), and 52 (defect width)/53 (defect depth) are arranged in increasing order of specificity. For example, row 51 lists selectable options as the anatomic subunits: boxes 51a refers to upper cutaneous lip (labeled in FIG. 5 as “UC”); 51b to lower cutaneous lip (LC); 51c to Philtrum (P); to nasal sill (NS). Likewise, boxes 52a, 52b, 52c, and 52d, respectively, refer to increasing defect widths, e.g., 10-33%. In addition, row 53 (defect depth) provides boxes labeled 53a-c, where 53a refers to supramuscular (SM); 53b to intramuscular; and 53 c to full thickness (FT).

In the example, as reflected in FIG. 5, a selection of Lip at box 50a prompts a series of choices from which box 51e (lower vermilion, one of the anatomical subunits) was selected according to the example. This selection prompted further choices about defect width and depth, respectively, leading to selections of 10-33% at box 52b and intramuscular at box 53b. With combination of these selections, the system according to the present example referred to a Vermilion island pedicle advancement flap at box 54. In some embodiments, box 54 may show multiple repair options depending on different parameters, with each option presented providing hyperlinks to additional information about the flap. This may include, as a non-limiting example, a readily accessible gallery of before and after photos for the same repair list is presented which can also be shown to the patient. Additionally, these photos serve as an educational or consultative resource to facilitate the discussion about which repair option might be best for the patient.

By designating particular details including the anatomic area, the surgeon also can access general tips for repairing defects within this anatomic area. Alternatively, the surgeon can view a list of all the repair options for that anatomic area without inputting any patient details, for example by clicking on “See all options” or similar designation within the system. Additionally, a surgeon can search a specific repair for a case by using the “Search repair options” bar, as seen in FIG. 2B, for example.

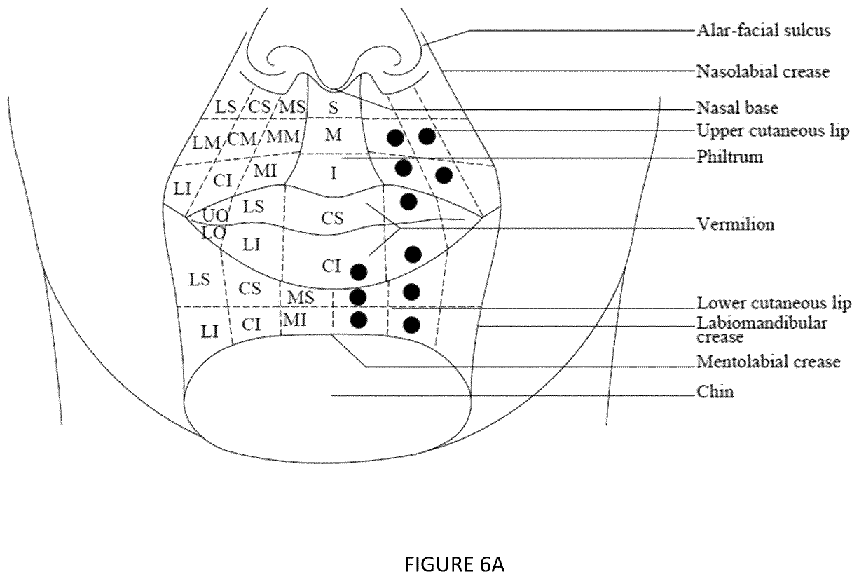

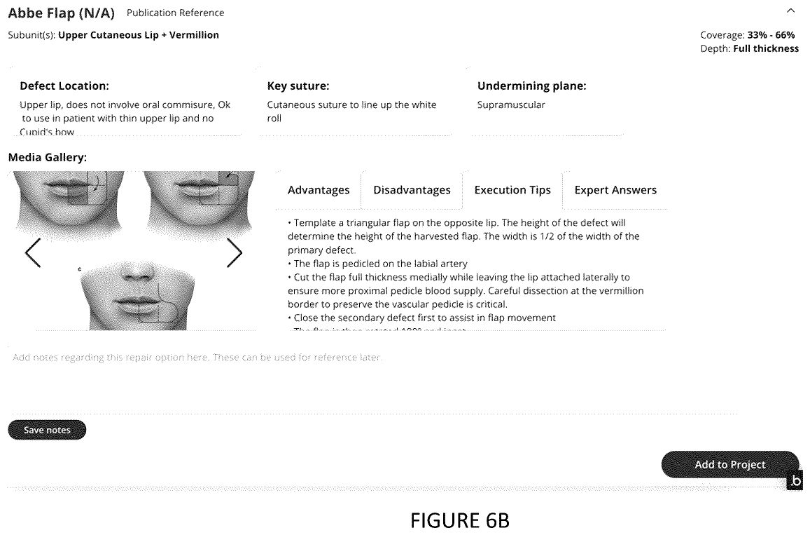

Generally in the practice of present embodiments, a surgeon will have access to facial diagrams (“Location Diagram), such as in FIG. 6A, which visibly illustrate all the anatomic/subunit areas where a specific flap may be employed, for example an Abbe flap designated in appropriate regions by a solid black dot in this figure. The Location Diagram can be viewed in the media gallery once a selection of a specific flap is made. The selection of the flap is made through a drop-down from the list of curated repair options. In addition to such a Location Diagram, once a selection of the flap is made, detailed information about a specific flap becomes available, for example the descriptions of an Abbe flap contained in FIG. 6B, which provides a screenshot of the kind of information available to a surgeon upon selecting a specific flap. Here, the selection is Abbe flap, and the output shown in the figure includes: appropriate defect location the flap can be used for, the key suture, the undermining plane, advantages, disadvantages, execution tips, and media gallery (which includes Location Diagram, photos, and videos). Additionally, there is a section for a surgeon to add notes and view previous questions about the flap and answers from expert surgeons. One can also click a link to a Publication Reference that further provides additional detail about the flap. If the user has a project in progress, from this screen the user can modify the project in real time, without having to save a new project by adding the flap to their existing project.

Users can further confirm that a flap of interest is intended to be used for a patient defect with specific parameters by looking at the diagram associated with the flap, an example shown in FIG. 6A. FIG. 6A represents all the possible sub-subunit locations of the lip where an Abbe flap can be used. The left side of FIG. 6A (i.e., the right side of the face shown) uses an abbreviation system in which L is for lateral and C is for central in a left to right context, and S is for Superior and I is for Inferior in an up to down context. In a left to right context, M is for medial, and in an up to down context, M is for middle. Thus, and by way of example, “CM” stands for central middle sub-subunit of the upper cutaneous lip subunit, and “LI” stands for lateral inferior of the lower cutaneous lip subunit. Accordingly, FIG. 6A denotes certain sub-subunits (without intending to be exhaustive) using small boxes bounded by dashed lines, and the same sub-subunits exist on both sides of the face (although only labeled on one side). Further, reference lines in FIG. 6A generally denote up to 7 sub-subunits (moving left to right). In this way, a Location Diagram allows greater specificity when the surgeon is determining all possible locations where a flap can be used. Again, regardless of the anatomical level (unit, subunit, or sub-subunit), the practice of present embodiments makes readily available in one place information that may include, by way of non-limiting example, key suture, undermining plane, advantages, disadvantages, execution tips, expert answers, flap diagrams, photos, videos, and links to reference articles.

Similar to FIG. 4, FIG. 7A lists repair options specific to known parameters based on patient findings, such as those discussed in connection with FIG. 4. FIG. 7B shows a different portion of this list providing other repair options. In this example, the lip remains the anatomic unit, the upper cutaneous lip remains the subunit, and the depth remains full thickness. However, the repair option is in reference to a defect size of 66-100%, resulting in different repair options as shown on the left in FIG. 7A.

As mentioned previously, a feature within the scope of present embodiments is that the surgeon can electronically link smart glasses to the system or platform and access this content for later use pre- or intraoperatively, allowing him or her to view diagrams and videos to help design and/or execute a flap in real time. Smart glasses include, but are not necessarily limited to, wearable eyeglasses that integrate computing capabilities, such as cameras, displays, and microphones, as well as monoscopic or stereoscopic virtual reality headsets, and similar viewing technologies capable of presenting a transparent digital display that overlays information onto real world observable conditions for a user, and providing hands-free access to digital information and connectivity. In some embodiments, paired smart glasses are connected to a monitor in the operating room or a remote monitor to allow viewers to see the reconstructive surgery as the surgeon is seeing it, optionally in real time or as a recording. In addition to other benefits, the use of smart glasses may also provide ready access to a comprehensive and curated list of repair options that are specific to the details of a patient's defect. In some embodiments, cameras suitable for use with a platform according to the present embodiments may include, but would not necessarily be limited to, a commercially available camera configured to capture photos and videos, including upon user command; voice assistant; and visual representations providing contextual information about a repair, such as but not limited to surgical navigation.

Notably, inventive systems according to present embodiments provide surgeons the ability to create and open a project, which identifies lists of specific flaps and repair techniques. projects can contain content about appropriate use, as well as patient-specific plans that incorporate the techniques from an existing project that a surgeon has saved, or patient-centric adjustments based on flap type, anatomic location or in this case subunit, or other factors the surgeon may make for a specific case, which then can be available for later use on upcoming surgical cases or “favorited” for future consideration. The surgeon is able to open a project to incrementally modify and save the content under a project by adding specific steps, annotations, photographs, measurements, and findings. Users can also use the platform to ask questions regarding a repair option and get potential answers from expert surgeons. Additional resources the system provides to the surgeon include, for example, bandaging techniques following wound closure. Access to such resources would work the same for bandaging as for other items such as flaps and repair options.

Consider, for example, a surgeon whose patient presents with a melanoma in situ on the cheek. The surgeon removes the melanoma in situ, then waits for pathologic confirmation that the entire skin cancer has been cleared. During this waiting period, the surgeon may use the embodiments of the present disclosure to plan out possible repair options in consultation with his or her patient. To accomplish this, the surgeon logs into the system, selects “cheek” as the anatomic location, selects the appropriate sub-anatomic location, and inputs the specific details about the patient's defect. The system then provides the surgeon with a list of possible repair options, which the surgeon reviews to decide which ones might be suitable or best for the case. Optionally, the surgeon then saves one or more of these repair options to a project for this patient case. In this way, the present embodiments provide the surgeon with key insights and techniques about the flap(s) which appear most suitable for a case, a process that would otherwise take a surgeon hours to find by having to access multiple reference textbooks or journals via paid subscriptions as indicated.

Additional details that a surgeon may use to narrow down the best flap option may include age, blood thinners, health, post-operative support, smoking status, and patient activity level. While waiting for pathologic results and before finalizing the repair plan, the surgeon may research specific flaps by reading about the details of each repair option, viewing photographic or video content, and accessing reference articles. In doing so, the surgeon uses these additional materials to update the project based on any additional factors. The ability to open an existing project, modify the contents incrementally, and save these changes will save significant time and avoid to perform a new search each time an incremental change is made.

By use of the embodiments as described herein, the surgeon can also better prepare for consultation with the patient about the patient's appropriate repair options, the advantages/disadvantages, and can even show the patient before and after photos. The repair decided upon is then an informed decision involving both the surgeon and the patient. While this example focuses on one possible use case involving one anatomic defect, it will be appreciated that the embodiments of the present disclosure can be used in multiple ways. The example provided here is meant to illustrate but not limit the range of options for how the embodiments can be used.

Among many features and benefits provided by the present disclosure, such a system prompts the surgeon to specify characteristics of their patient's defect (i.e., anatomic location, size and depth) in order to receive a list of appropriate reconstructive options. In this way, searches and lists of options can be narrowed through various levels of selections, such as anatomic unit/body part (e.g., lip), followed by sub-units (e.g., philtrum), followed by sub-subunits (e.g., inferior or superior philtrum). Certain defects cross anatomic units, so in some embodiments the surgeon is able to select multiple units and access content for integration of repair techniques across multiple anatomic units to allow for even more tailoring of individual patient reconstructive plans.

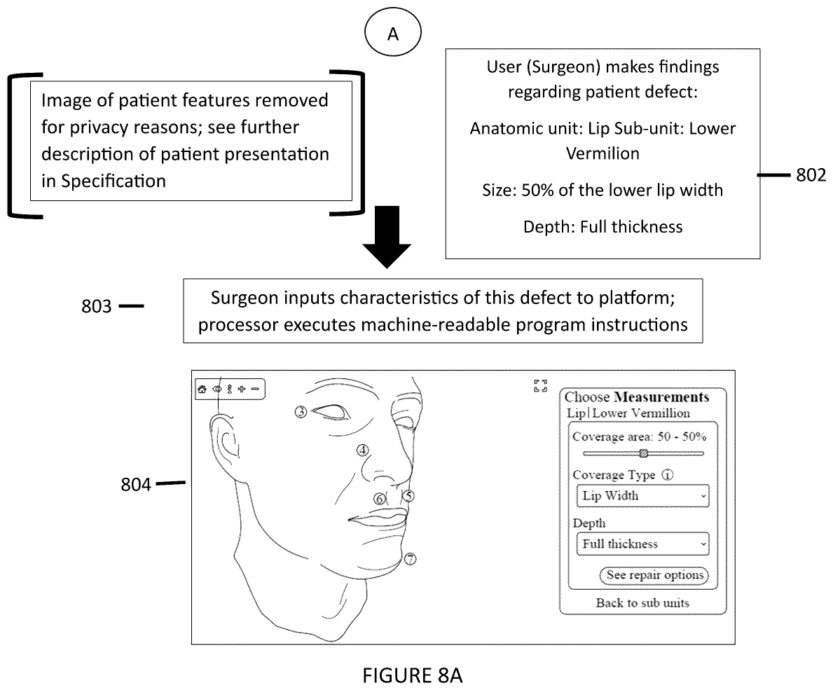

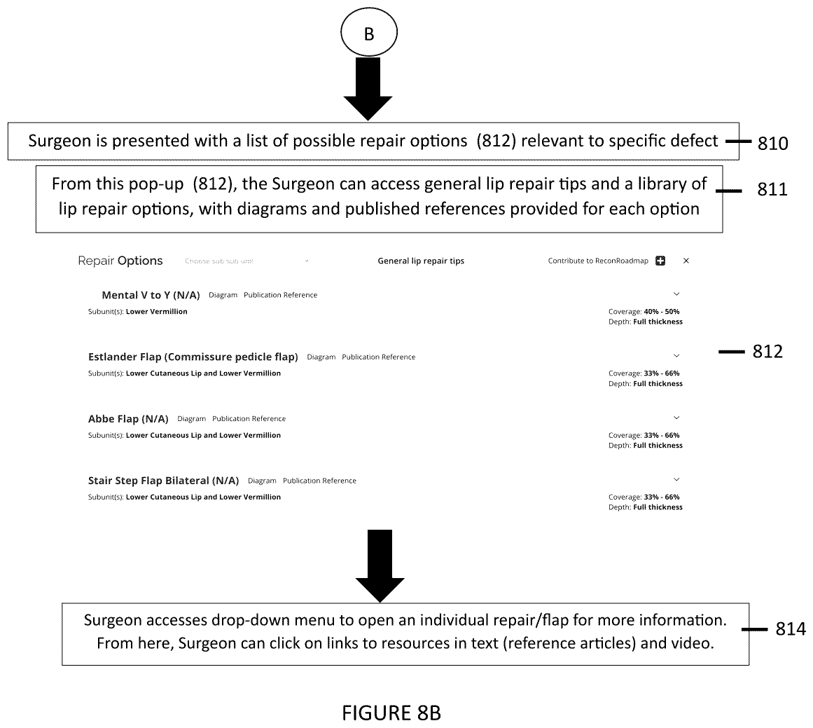

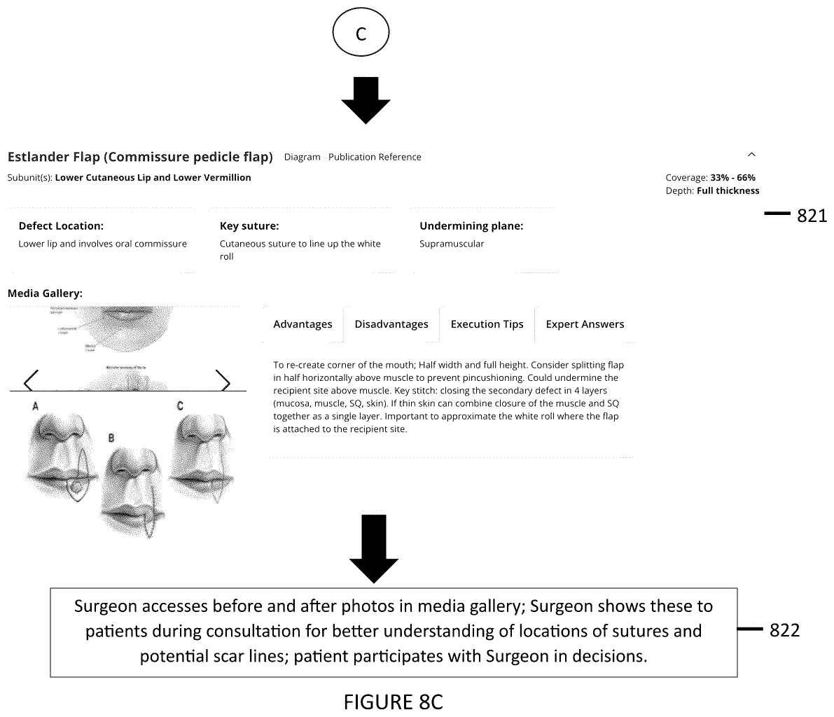

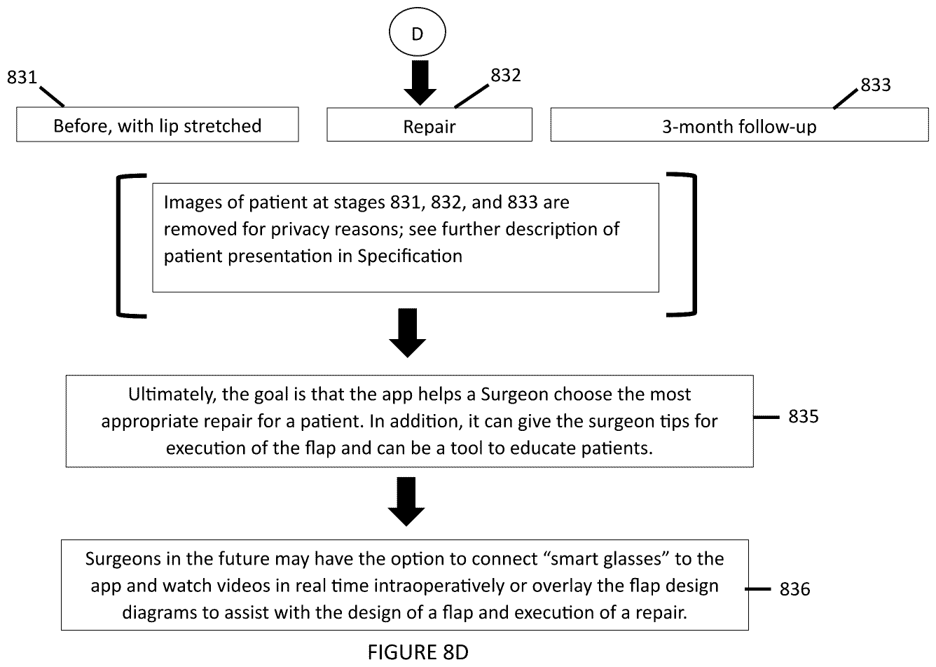

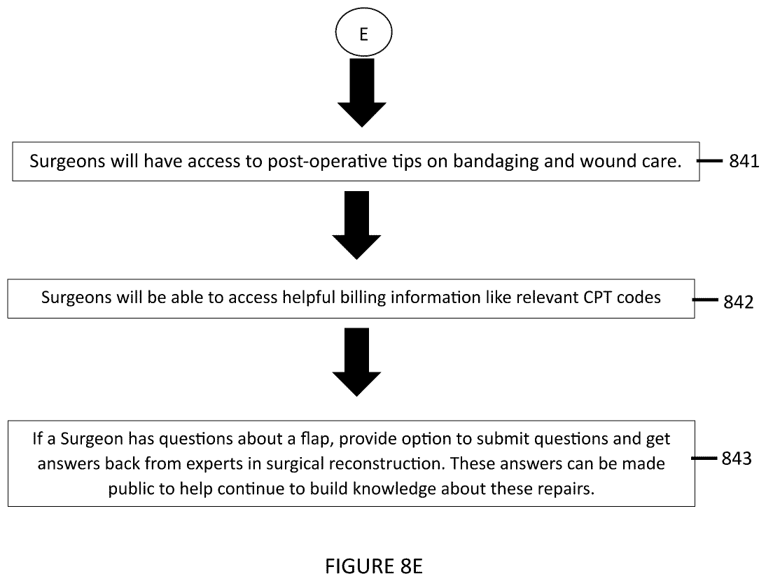

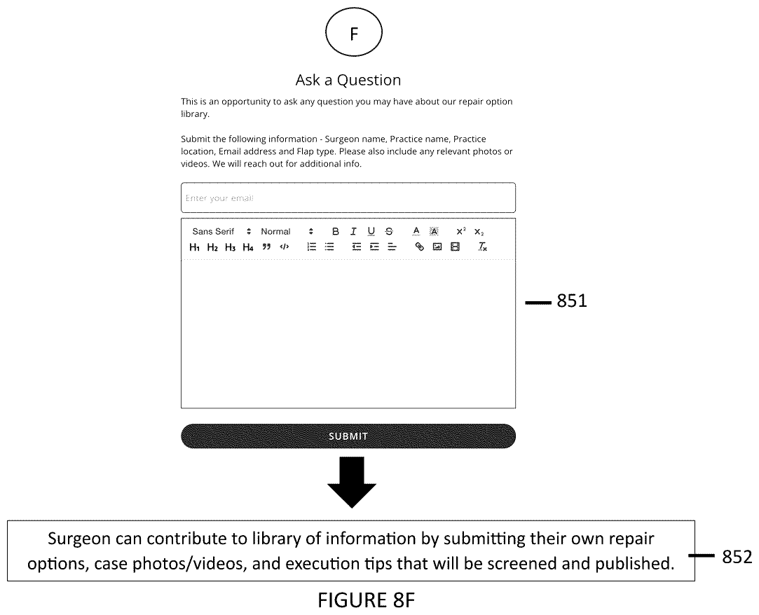

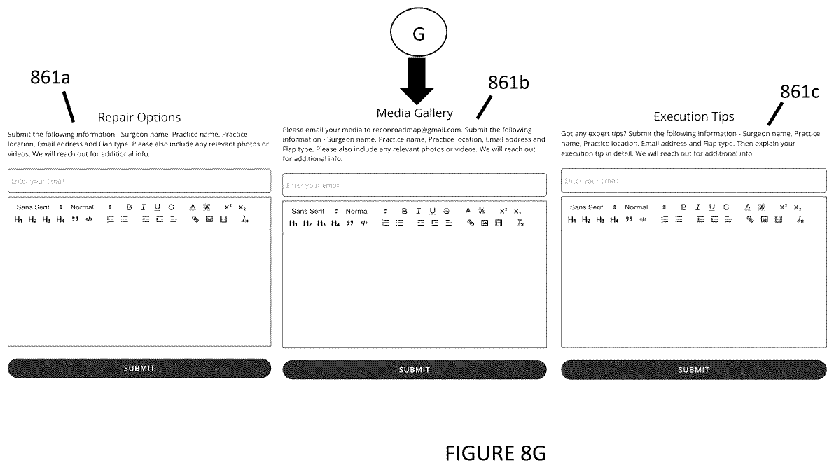

Turning back to the figures now, FIG. 8 (divided into parts 8A-8G) is a flow diagram that shows how a surgeon could incorporate this system into a regular work flow from cancer defect to flap options/design to intra-operative details to post-operative questions/concerns. Beginning with FIG. 8A, “A” marks the beginning of the flow, wherein at steps 802 and 803, respectively, findings obtained by the surgeon related to a defect are input to characterize the particular defect. As reflected in FIG. 8, the platform includes a processor operatively connected to memory, with the processor executing machine-readable program instructions related to the workflow. In this regard, the platform, i.e., system, may combine hardware and/or software code, routines, modules, or instructions configured to be executed and perform the steps and functions described herein. The instructions executed by the processor may be stored in memory operatively connected to the processor. Such memory may include, but is not necessarily limited to volatile or non-volatile memory, computer readable storage media such as compact disc, DVD, hard drive, flash memory, random access memory (RAM), read only memory (ROM) or universal serial bus (USB)-based drive, to name some.

In some embodiments, a displayed user interface represented as screen shot 804 enables this input. At point “B” as shown in FIG. 8B, the process continues wherein the surgeon is provided repair options at step 810, which may also encompass additional resources as mentioned in step 811. Exemplary flap options for a particular defect are shown in screen shot 812, allowing for access to additional information, articles, video, and other resources, step 814. Turning to FIG. 8C, screen shot 821 illustrates a webpage with additional resources the surgeon can access. In turn, step 822 reflects an ability to use various images, including but limited to before and after photos, during patient consultations, enabling more focused participation by the patient in the care decisions.

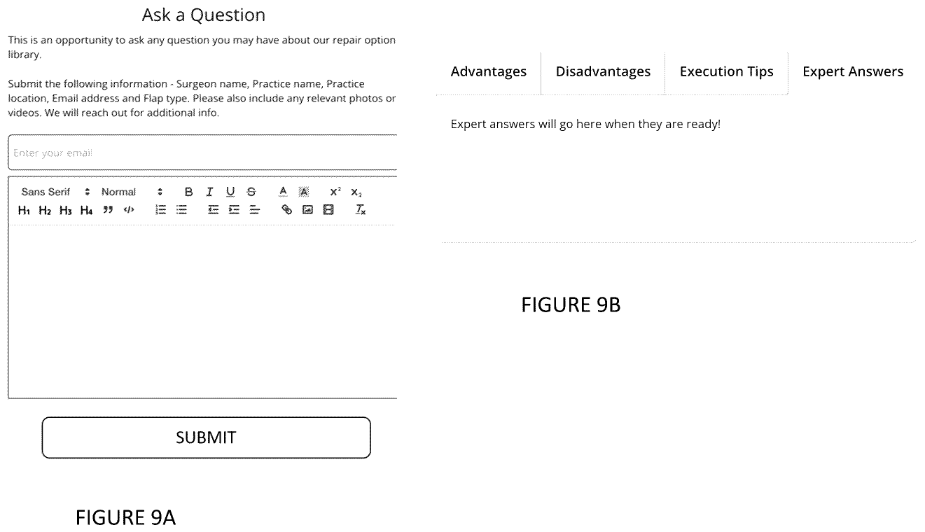

In planning a particular project, photos at various points obtained from example cases (appropriately consented) or publications can be included to visually show a patient how their flap may be expected to look immediately after execution and then a few weeks to months into healing. In this respect, FIG. 8D continues the flow diagram by describing de-identified patient-specific folders with photos before the repair (831), the repair itself (832), and follow-up photos (833). At step 835, the surgeon continues to choose the most appropriate repair in consultation wit the client. At step 836, an optional feature using smart glasses can be employed in visualizing the surgical repair potentially as a virtual overlay on the patient. FIG. 8E references additional resources provided by the platform, including post-operative considerations at step 841, billing and codes at step 842, and answer to specific questions at step 843. In some embodiments, as FIG. 8F shows, questions can be submitted and responded to by a fellow surgeon or other expert at step 851. In turn, answers to submitted questions can be curated as part of an expanding library of information, including saved answers to relevant questions at step 852, and saved as shown in FIGS. 9A, 9B. Optionally, as FIG. 8G illustrates, expert surgeons can submit information organized in categories for contribution. Such categories may include repair options (861a), media gallery (861b), and execution tips (861c), for example.

In an exemplary case, suppose an image of the lower face of a patient, as depicted in screen shot 804 in FIG. 8A, showed a significant wound that affecting about 50% of the lower lip of the individual. Suppose further the wound was a post-operative wound after Mohs micrographic surgery, appearing as an open, ulcerated lesion, exposing muscle. Now turning to FIG. 8D, images associated with the “Before” stage (box 831) for this particular patient would be expected to show a significant lower lip wound, exposing muscle and comprising about 70% of the lower vermilion and cutaneous lip. Following an Estlander transposition flap, images associated with the “Repair” stage (832) would show suture lines in the lower face running from the right nasolabial fold at the superior apical lip to the right corner of the mouth. Suppose further this flap itself was an upside-down triangle-shape running from the right corner of the mouth to the right mental crease and then back up to the vermilion border at the center of the lower lip. FIG. 8D (box 833) would provide for submission of images at a “3-month follow-up stage,” as an example patient using this flap.

In another exemplary flap design executed on a patient by the inventors, a top-down view of the patient's scalp would show a surgical wound in the center of the top of the head. Surgical markings were drawn in an S-like pattern to resemble a spiral design to outline the placement for a flap reconstruction, particularly to indicate where incision lines were made. These incisions allowed the flaps to be released and brought together in order to cover the surgical wound in the center. In another exemplary surgery, a different patient underwent a repair of a post-Mohs micrographic surgery wound on the skin in front of the right ear of a different patient, where the wound measured about 2.5×2.0 cm. Again, markings were used around the wound to represent planned incisional lines for a Burow's wedge advancement flap. In particular, a marking comprising a vertical line was made in the preauricular crease, with a triangular cone drawn at the supraauricular crease, with a second triangular cone drawn at the infraauricular crease extending behind the ear. The triangular cones represented where tissue will be removed and discarded, as once the flap is advanced into place to cover the wound, these cones will be considered excess tissue. It will, therefore, be appreciated that the surgical planning and consultation in regards to the patients discussed in relation to FIG. 8, parts A and D, along with the above two exemplary cases were facilitated through the practice of a surgical reconstructive planning system, according to multiple embodiments as described herein. In turn, FIG. 8, parts B, C, E-G further delineate exemplary steps and resources that may be accessed in uses cases such as those discussed herein.

From a wider perspective, a surgical reconstructive planning system as described herein also is intended to serve as a growing and changing dynamic repository of the kinds of content described herein. For example, expert surgeons, with expressive consent can also submit original flap designs, new applications of existing flaps, execution tips, and flap photos and videos to continue to add to the stored repository contained in the inventive system. Such contributions will offer more immediate access to peers compared to publishing in a textbook or journal, which may take months or even years, but will still be vetted for suitable quality.

Also by way of example, and in FIGS. 9A (shown on the left, which contains the same content as box 851 in FIG. 8F) and 9B (on the right), surgeons may ask questions to vetted experts in the field, either in the form of or during the pre-planning and post-operative stages. As these questions and tips are provided to the individual surgeon, the content can be further curated and added to the system's storage in the form of a library for access by other surgeons. This will allow surgeons to access previously asked and answered questions regarding each specific flap, thereby saving significant time that otherwise would be required for research. For example, less experienced surgeons can ask specific questions regarding a particular flap that more experienced surgeons may answer. In addition, highly qualified surgeons can contribute to the continuously growing body of knowledge around flap techniques by submitting repair options, execution tips, photographs and videos from procedures, and the like. While current embodiments provide these capabilities, users are solely responsible to ensure that any use of such a system, platform, or method described complies with all applicable patient privacy laws and intellectual property laws. For the avoidance of doubt, the owners of the subject matter described herein bear no responsibility for user compliance with applicable patient privacy laws and intellectual property laws.

Other content that is stored in the system and accessed by surgeons may include indicators and suggestions for billing based on flap/repair and anatomic area, especially for common CPT codes. To the inventors'knowledge, this is the first resource for reconstructive surgery that is interactive, collaborative, project-based, iteratively adjustable from project to project or within the same project, while continuing to expand in content. A user can save particular flaps as projects, which they have used or intend to use, and provide annotations or other modifications to their projects over time. This allows a project to be saved, opened, modified, and accessed again and again as the project evolves over time. This can be particularly helpful given that information known about a patient's condition and the particular defect in need of repair can change during the time between the initial consultation and the surgery. The flexible, modifiable nature of the projects in response to new developments or techniques will save significant time by keeping the resource information, as well as the surgeon's personal planning for a specific patient, in one comprehensive place. Intended users and beneficiaries will include, but not necessarily be limited to, reconstructive surgeons, novice surgeons, their staff, and their patients.

Accordingly, a surgical reconstructive planning system or method in accordance with present embodiments may comprise a storage device configured to store a plurality of images or video recordings of or related to a plurality of flap repairs, and arranged in a manner as chosen by a user such as by anatomic location, type of repair, or extent of defect to name some. Such images and video recordings may be taken from different angles from one or more exemplary patients. The storage device can be further configured to store a set of visual objects representing diagrams, instructions, tips, annotations, or other subject matter related to surgical reconstructive options. Additionally, a user interface may be configured to allow a surgeon to view the plurality of images or video recordings and the set of visual objects in a viewing window, and to create a project by accessing the viewed images or video recordings and visual objects. Such a user interface may also be linkable with electronic devices used by a surgeon intraoperatively, including but not limited to smart glasses. Upon allowing a surgeon to view the images, video recordings, and visual objects, the user interface may allow the surgeon to select at least one of the plurality of images or video recordings and at least one of the visual objects to include in a project. Systems according to the present disclosure herein may further be comprised of a processor, executing machine-readable instructions in communication with the user interface and display, enabling the surgeon to preview the project, and then to modify the project by either adding, removing, or reordering at least one of the images, video recordings, or visual objects. In this way, the processor enables the project to be saved, reopened and iteratively modified by at least one of the following: adding, removing, or reordering at least one of the images, video recordings, or visual objects. The project can be designated as finished or “in progress” for continual use before, during, and after reconstructive surgery.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of such words and phrases as “such as,” “comprising,” “e.g.,” “containing,” or “having” and variations of those words is meant to encompass the items listed thereafter, and equivalents of those, as well as additional items. The use of “including” (or, “include,” etc.) should be interpreted as “including but not limited to.”

Accordingly, the foregoing descriptions of several embodiments and alternatives are meant to illustrate, rather than to serve as limits on the scope of what has been disclosed herein. It will be understood by those having ordinary skill in the art that modifications and variations of these embodiments are reasonably possible in light of the above teachings and descriptions.

What is claimed is:

1. A surgical reconstruction planning method for a cutaneous defect using a digital platform, comprising:
providing access via the platform to first electronically stored information comprising a list of one or more reconstructive options for reconstruction of the cutaneous defect;
accepting a user selection via the platform of a reconstructive option, wherein said reconstructive option selection prompts a display of textual or graphic information about the selected reconstructive option;
providing access via the platform to second electronically stored information comprising a set of visual objects selectable by a user representing content related to at least one of the reconstructive options;
accepting a user selection via the platform of at least one of the visual objects;
arranging the one or more visual objects in a preview window viewable by the user according to a first arrangement, wherein said arrangement includes a sequence in which the visual objects appear;
enabling the first arrangement of visual objects to be saved as a stored electronic file referred to as a project; and
enabling the saved project to be opened and reopened and modified by at least one of adding visual objects to the project, removing visual objects from the project, rearranging the sequence of visual objects in the project, and adding patient-specific measurements, photographic or video images, or annotations related to the project.

2. The method of claim 1, wherein one or both of the first and second electronically stored information are obtained from a digital library operatively connected to the platform.

3. The method of claim 1, wherein the visual objects comprise at least one of anatomic diagrams, anatomic photographs, surgical procedure videos, and flowcharts.

4. The method of claim 1, wherein the platform is accessed through an internet connection.

5. The method of claim 1, wherein the visual objects comprise at least one of anatomic diagrams, anatomic photographs, surgical procedure videos, and flowcharts directed to one or more of the reconstructive options available for user selection.

6. The method of claim 1, wherein the reconstructive options include textual or graphic information appearing in the preview window.

7. The method of claim 1, wherein the second electronically stored information is arranged as a hierarchical data structure operatively connected to the platform.

8. The method of claim 1, further comprising providing an electronically paired connection between the platform and smart glasses.

9. The method of claim 8, wherein the smart glasses comprise a transparent digital display that overlays information onto real world observable conditions.

10. The method of claim 9, wherein a visual representation providing information about a repair is electronically paired to the display of the smart glasses.

11. The method of claim 9, further comprising configuring a camera to capture photos and videos upon user command and configuring the project to receive the captured photos or videos.

12. An at least partially automated method for planning a surgical reconstruction of a cutaneous defect from multiple reconstructive options, comprising:
electronically storing first electronically stored information comprising one or more reconstructive options for repairing the cutaneous defect;
electronically storing second electronically stored information comprising one or more visual objects related to the reconstructive options;
accepting user input of one or more factors related to the cutaneous defect;
automatically identifying a selection of at least one reconstructive option for repairing the cutaneous defect from the first electronically stored information;
executing machine readable program instructions on a processor to associate the at least one identified reconstructive option with one or more visual objects obtained from the second electronically stored information;
arranging the one or more associated visual objects, wherein said arrangement includes a sequence in which the visual objects appear;
displaying the arranged visual objects in a preview window viewable by the user after selection;
enabling the sequence of visual objects to be saved as a stored electronic file referred to as a project; and
enabling the saved project to be opened and reopened and modified by at least one of adding visual objects to the project, removing visual objects from the project, rearranging the sequence of visual objects in the project, and adding patient-specific measurements, photographic or video images, or annotations related to the project;
wherein the automatic selection of the reconstructive option is accompanied by a display of textual or graphic information about the selected reconstructive option viewable by a user in a preview window.

13. The method of claim 12, wherein one or both of the first and second electronically stored information are obtained from a digital library.

14. The method of claim 12, further comprising arranging a digital platform and storing the first and second electronically stored information on the platform, wherein the platform is accessed from the internet.

15. The method of claim 12, wherein the one or more factors are chosen from the group location, size and depth of defect.

16. The method of claim 15, wherein location is a position on the lip of a patient and is chosen from the group philtrum, upper and lower cutaneous lip, upper and lower vermilion, oral commissure, and nasal sill.

17. The method of claim 12, wherein the visual objects comprise at least one of anatomic diagrams, anatomic photographs, surgical procedure videos, and flowcharts.

18. A surgical reconstruction planning method for a cutaneous defect using a digital platform, comprising:
accepting user input of one or more factors related to the cutaneous defect;
providing access via the platform to a list of one or more reconstructive options for reconstruction of the cutaneous defect;
accepting a user selection via the platform of a reconstructive option, wherein said reconstructive option selection prompts a display of textual or graphic information about the selected reconstructive option;
wherein accepting a user selection comprises automatically identifying a selection of at least one reconstructive option for repairing the cutaneous defect from the list of options and allowing the user to confirm the identified selection or select a different option from the list;
providing access via the platform to two or more visual objects selectable by a user representing content related to at least one of the reconstructive options;
accepting a user selection via the platform of at least one of the visual objects;
arranging the one or more visual objects in a preview window viewable by the user according to a first arrangement, wherein said arrangement includes a sequence in which the visual objects appear;
enabling the first arrangement of visual objects to be saved as a stored electronic file referred to as a project; and
enabling the saved project to be opened and reopened and modified by at least one of adding visual objects to the project, removing visual objects from the project, rearranging the sequence of visual objects in the project, and adding patient-specific measurements, photographic or video images, or annotations related to the project.

19. The method of claim 18, wherein the visual objects comprise at least one of anatomic diagrams, anatomic photographs, surgical procedure videos, and flowcharts directed to one or more of the reconstructive options available for user selection.

20. The method of claim 18, further comprising providing an electronically paired connection between the platform and smart glasses.

21. The method of claim 20 wherein the smart glasses comprise a transparent digital display that overlays information onto real world observable conditions, and a visual representation providing information about a repair is electronically paired to the display of the smart glasses.

22. The method of claim 21, further comprising configuring a camera to capture photos and videos upon user command and configuring the project to receive the captured photos or videos.
